(19) United States
(12) Patent Application Publication
(10) Pub. No.: US 2006/0281817 A1
(43) Pub. Date: Dec. 14, 2006

(54) Treatment for neurodegenerative diseases

(76) Inventors: Carol A. Colton, Cary, NC (US); Michael P. Vitek, Cary, NC (US); Warren J. Strittmatter, Hillsborough, NC (US); Qing Xu, Durham, NC (US); James R. Burke, Chapel Hill, NC (US)

Correspondence Address: MYERS BIGEL SIBLEY & SAJOVEC, PO BOX 37428, RALEIGH, NC, 27627, US

(21) Appl. No.: 11/448,260

(22) Filed: Jun. 7, 2006

Related U.S. Application Data
(60) Provisional application No. 60/688,491, filed on Jun. 8, 2005.
(60) Provisional application No. 60/722,152, filed on Sep. 30, 2005.

(51) Int. Cl. A61K 31/198; A61K 31/13

(52) U.S. Cl. 514/565; 514/673; 514/674

(57) ABSTRACT

A method of treating a neurodegenerative disease in a subject in need thereof comprises: (a) administering the subject an ornithine decarboxylase inhibitor such as alpha-difluoromethylornithine or a pharmaceutically acceptable salt or prodrug thereof in an amount effective to treat the disease; and concurrently (b) administering the subject a polyamine or a pharmaceutically acceptable salt or prodrug thereof in an amount effective to reduce damage to the gastroinestinal tract of the subject by the ornithine decarboxylase inhibitor.

### RELATED APPLICATION

This application claims the benefit of U.S. Provisional Patent Application Ser. No. 60/688,491, filed Jun. 8, 2005, and of U.S. Provisional Patent Application Ser. No. 60/722,152, filed Sep. 30, 2005, the disclosures of both of which are incorporated by reference herein in their entirety.

This invention was made with U.S. Government Support under Grant No. AG19740 from the National Institutes of Health. The U.S. Government has certain rights to this invention.

### FIELD OF THE INVENTION The present invention concerns methods and compositions useful for the treatment of neurodegenerative diseases.

### BACKGROUND OF THE INVENTION

The polyglutamine-repeat diseases, a group of at least eight disorders, result from the expression of mutant proteins having in common an expanded polyglutamine domain. Huntington's disease (HD) is one autosomal dominant polyglutamine-repeat disease. HD is a progressive, fatal neurodegenerative disorder that displays characteristic abnormal motor functions, behavior changes and dementia. There are presently no satisfactory treatments and there continues to be a need for new ways to treat neurodegenerative diseases such as HD.

### SUMMARY OF THE INVENTION

A first aspect of the present invention is a method of treating a neurodegenerative disease in a subject in need thereof. The method comprises: (a) administering, preferably orally administering, the subject an ornithine decarboxylase inhibitor such as alpha-difluoromethylornithine or a pharmaceutically acceptable salt or prodrug thereof in an amount effective to treat the disease; and concurrently (b) administering, preferably orally administering, the subject a polyamine or a pharmaceutically acceptable salt or prodrug thereof in an amount effective to reduce damage to the gastroinestinal tract of the subject caused by the ornithine decarboxylase inhibitor.

A second aspect of the present invention is a method of treating a neurodegenerative disease in a subject in need thereof. The method comprises simultaneously orally administering in a single oral dose formulation: (a) an ornithine decarboxylase inhibitor such as alpha-difluoromethylornithine or a pharmaceutically acceptable salt or prodrug thereof in an amount effective to treat the disease; and (b) a polyamine or a pharmaceutically acceptable salt or prodrug thereof in an amount effective to reduce damage to the gastroinestinal tract of the subject caused by the ornithine decarboxylase inhibitor.

A third aspect of the invention is an oral dose formulation for treating a neurodegenerative disease comprising or consisting essentially of, in combination: (a) an ornithine decarboxylase inhibitor such as alpha-difluoromethylornithine or a pharmaceutically acceptable salt or prodrug thereof in an amount effective to treat the disease; and (b) a polyamine or a pharmaceutically acceptable salt or prodrug thereof in an amount effective to reduce damage to the gastroinestinal tract of the subject caused by the ornithine decarboxylase inhibitor. The formulation may be in any suitable form, such as a tablet, capsule or liquid.

A further aspect of the present invention is the use of an active agent as described herein for the preparation of a medicament for carrying out a method of treatment as described herein.

In a particular embodiment of the invention, the methods of the present invention are useful in the treatment of Parkinson's disease.

The foregoing and other objects and aspects of the present invention are explained in greater detail in the drawings herein and the specification set forth below.

### BRIEF DESCRIPTION OF THE DRAWINGS

FIG. 1 shows the timeline and protocol of a demonstration of the present invention in Huntington's disease (HD) mice.

FIG. 2 shows Average survival is increased by treatment with DFMO and putrescine in HD mice compared to untreated HD mice, in accordance with FIG. 1 (Average increased survival=33%; Averaged increased median survival=49%).

FIG. 3 shows the Kaplan-Meyer Survival curve for HD untreated-vs-HD treated mice treated in accordance with FIG. 1.

FIG. 4 shows that rotorod motor function is better in DFMO treated HD mice treated in accordance with FIG. 1.

### DETAILED DESCRIPTION OF PREFERRED EMBODIMENTS

Neurodegenerative disease as used herein includes, for example, amyotrophic lateral sclerosis (or ALS), Parkinson's disease, multiple sclerosis, and expanded polyglutamine repeat diseases.

Expanded polyglutamine repeat diseases (or “polyglutamine repeat diseases) with which the present invention is concerned include, but are not limited to, Huntington's disease, dentatorubral pallidoluysian atrophy, spinobulbar muscular atrophy, and spinocerebellar ataxia types 1, 2, 3, 6 and 7. See, e.g., U.S. Pat. No. 6,632,616 to Burke et al.

“Treat” as used herein refers to any type of treatment that imparts a benefit to a patient afflicted with a disease, including improvement in the condition of the patient (e.g., in one or more symptoms), delay in the progression of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

“Concurrent administration” means sufficiently close in time to produce a combined effect (that is, concurrently may be two or more events occurring within a short time period before or after each other). Concurrently is inclusive of “simultaneous.”

“Simultaneous administration” as used herein means that the active compounds are administered at the same point in time or immediately following one another. In the latter case, the two compounds are administered at times sufficiently close that the results observed are indistinguishable from those achieved when the compounds are administered at the same point in time.

“Chronic administration” as used herein refers to administration over a prolonged period of time, as opposed to acute administration in which administration is terminated when a disease is sufficiently treated or cured. For example, chronic administration may be administration multiple times a week (e.g., one, two or three times daily for three, four, five, six, or seven days a week) for a period of at least one, two, three, four, five or six months or more, after which time disease progression is reassesed by the treating physician and dosage is continued or adjusted as necessary, with treatment typically continuing on an ongoing basis.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention.

The present invention is primarily concerned with the treatment of human subjects, but the invention may also be carried out on animal subjects, particularly mammalian subjects such as mice, rats, dogs, cats, livestock and horses for veterinary purposes, and for drug screening, testing and development purposes.

The disclosures of all United States Patent references cited herein are incorporated by reference herein in their entirety.

1. Active Compounds.

Ornithine decarboxylase inhibitors are known and described in, for example, U.S. Pat. Nos. 5,753,714; 5,132,293; 5,002,879; 4,720,489; and 4,499,072. Examples include, but are not limited to, alpha-difluoromethylornithine, 2-(difluoromethyl)-2,5-diaminopentanoic acid; alpha-ethynyl ornithine; 6-heptyne-2,5-diamine; 2-methyl-6-heptyne diamine; alpha.-difluoromethyl ornithine; the methyl ester of monofluoromethyl dehydroornithine; the R,R-isomer of methyl acetylenic putrescine, 3-aminooxy-1-aminopropane (APA) and its analogs or derivatives such as CGP 52622A and CGP 54169A, 1,25-dihydroxycholecalciferol, and pharmaceutically acceptable salts and prodrugs thereof.

Additional examples of ornithine decarboxylase inhibitors that may be used to carry out the present invention include but are not limited to:

| Drug | Company |
|---|---|
| CGP-52622A | Novartis AG |
| CGP-54169A | Novartis AG |
| dihydroxycholecalciferol | Chugai Pharmaceutical Co Ltd |
| insulin analogs, Scios/Pfizer | Scios Inc |
| CGP-51905A | Novartis AG |
| CGP-45300A | Novartis AG |
| anticancer agents, University of | University of Illinois |
| Illinois | |
| vitamin D analogs (cancer), | Johns Hopkins University |
| Cytochroma/Johns Hopkins | |

Alpha-difluoromethylornithine (DFMO; eflornithine (sometimes called “elfornithine”) is known and described in, for example, U.S. Pat. Nos. 6,730,809; 6,573,290; 6,258,845; and 4,925,835.

Polyamines useful for carrying out the present invention are known and described in U.S. Pat. Nos. 6,867,237; 6,794,414; 6,368,598; and 5,885,982. Polyamines may be primary amines, including C1-C5 alkyl amines. Examples of suitable polyamines include but are not limited to spermine, spermidine, putrescine (1,4-diamino-butane), 1,3-diamino-propane, 1,7-diamino-heptane, and 1,8-diamino-octane.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Prodrugs of active compounds as noted above are in general compounds that are rapidly transformed in vivo to yield the desired active agent. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. Nos. 6,680,324 and 6,680,322.

2. Formulations and Administration.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Capsules may contain particles coated with the active agents. Particles used herein may be of any suitable size, typically from about 100 to 1000 micrometers in diameter. Examples include particles with a diameter of about 600 to 250 um (30-60 mesh), or a diameter of 700 to 600 um (25-30 mesh). Size of particles can be determined in accordance with known techniques, such as described in the CRC Handbook, 64^{th} edition, page F-114 and USP24/NF19, page 1969. Any suitable core material can be used for the particles. Examples of such materials are polymers e.g., plastic resins; inorganic substances, e.g., silica, glass, hydroxyapatite, salts (sodium or potassium chloride, calcium or magnesium carbonate) and the like; organic substances, e.g., activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides. In addition, the particles according to the present invention may further contain one or more additional additives such as thickening agents, lubricants, surfactants, preservatives, complexing and chelating agents, electrolytes or other active ingredients, e.g., antiinflammatory agents, antibacterials, disinfectants or vitamins. The particles may be inserted into a suitable capsule such as a gelatin capsule in accordance with known techniques.

Tablets can be produced by conventional tabletting techniques with conventional ingredients or excipients. The tablets are preferably formed from a composition comprising the particles described herein distributed in a mixture of a disintegrant and a diluent or filler. Suitable disintegrants include, but are not limited to, crospovidone and croscarmellose. Suitable diluents include, but are not limited to, lactose, sucrose, dextrose, mannitol, sorbitol, starch, cellulose, calcium phosphate, microcrystalline celulose such as AVICEL™, etc. Tablets may include a variety of other conventional ingredients, such as binders, buffering agents, lubricants, glidants, thickening agents, sweetening agents, flavors, and pigments.

Liquids can be prepared in any suitable form, such as a syrup, in accordance with known techniques by dissolving, solubilizing or suspending (e.g., as an emulsion or microemulsion) the active agents therein. Such liquids may be aqueous liquids, optionally including additional cosolvents such as oils and the like.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. In general, the alpha-difluoromethylornithine is included in the formulation or administered to the subject in an amount effective to deliver a dosage thereof of 0.1 or 0.5 grams per meter² per day to the subject, up to 2, 3 or 5 grams per meter² per day to the subject. In general, the polyamine is included in said formulation or administered to the subject in an amount effective to deliver a dosage thereof of 20, 50 or 100 mg per kg per day to the subject, up to 1000 or 2000 mg per kg per day to the subject.

The present invention is explained in greater detail in the following non-limiting Examples.

### EXAMPLE 1

Huntington's disease (HD) mice are transgenic mice that express a cDNA encoding an N-terminal fragment (171 amino acids) of huntingtin with 88 glutamine repeats. HD mice are known. See, e.g., G. Schilling et al., Intranuclear inclusions and neuritic aggregates in transgenic mice expressing a mutant N-terminal fragment of huntingtin, Hum. Mol. Genet 8(3), 397-407 (1999).

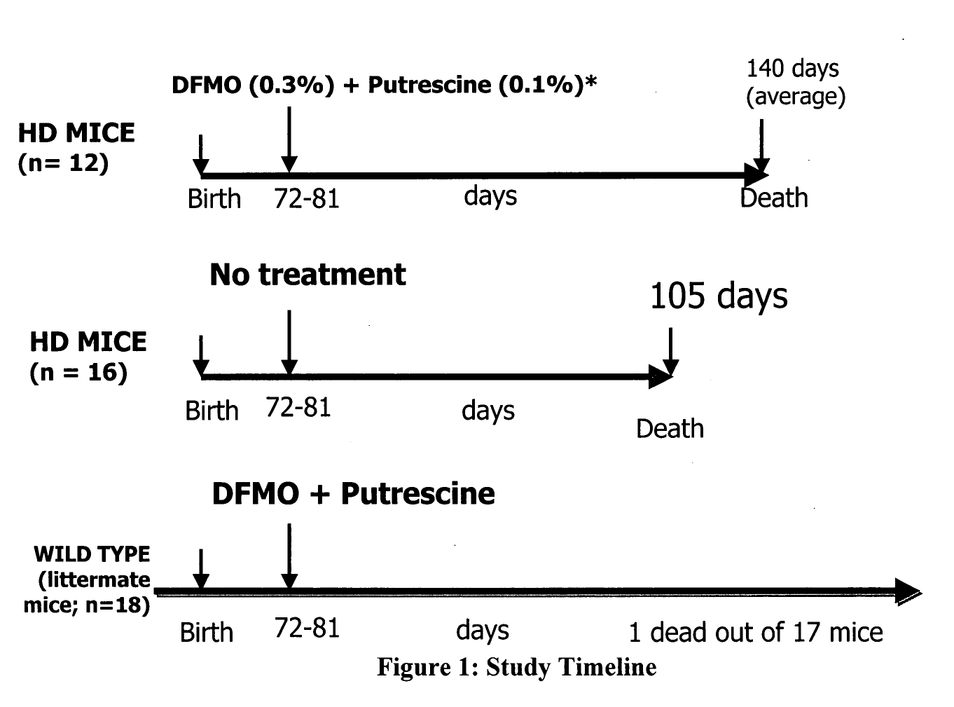

This example was carried out as schematically illustrated in FIG. 1. Briefly, experimental HD mice (n=12) were administered DFMO (0.3%) plus putrescine (0.1%) added to their drinking water for a period beginning 72 days after birth through 81 (199) days after birth or until death of the animal. Control HD mice received the same food and water but without added DFMO plus putrescine. Control wild-type mice received DFMO plus putrescine in like manner as the experimental mice.

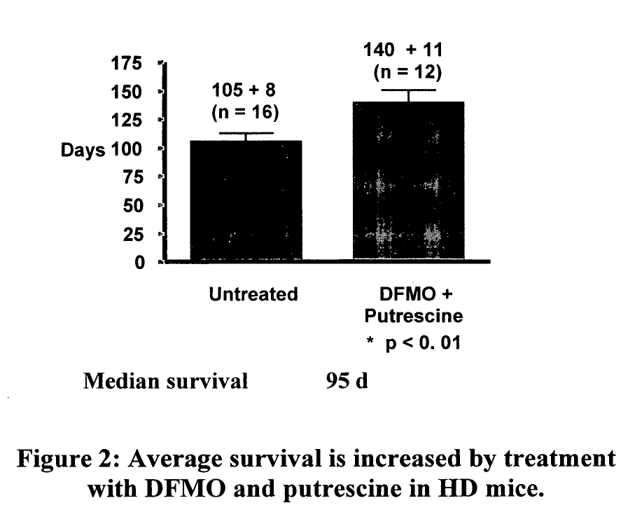

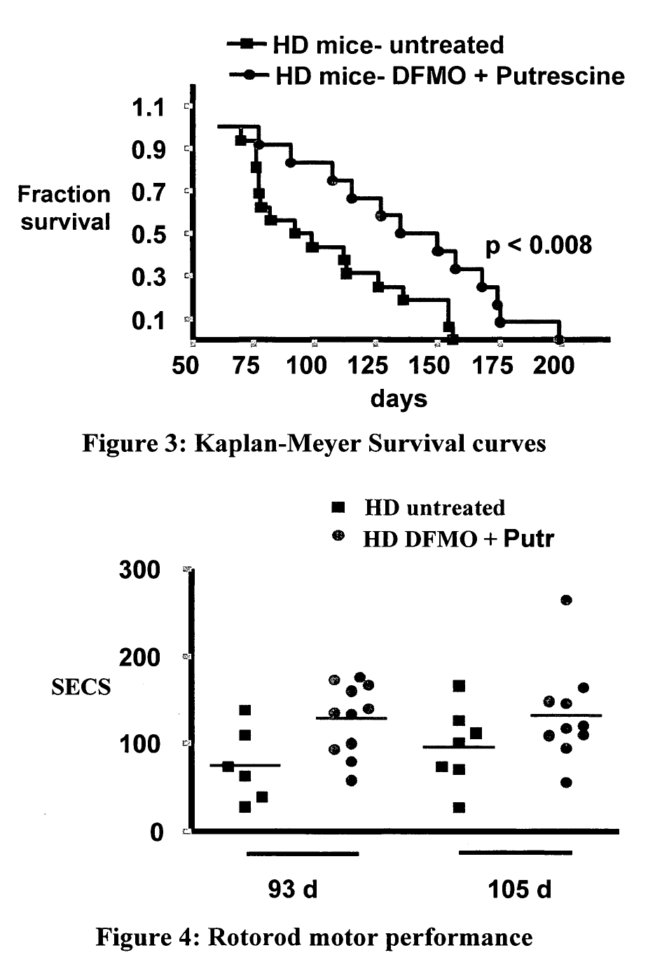

As shown in FIG. 2, the average survival of HD receiving experimental mice (right bar) was substantially increased as compared untreated HD mice (left bar). The average increased survival was 33% and the averaged increased median survival was 49%) Likewise, FIG. 3 shows that the Kaplan-Meyer Survival curve for HD untreated-vs-HD treated mice, with the curve substantially improved for the treated mice.

As shown in FIG. 4, motor function, as determined by the rotorod test, was improved in DFMO plus putrescine treated HD (circles) mice as compared to untreated HD mice (squares) at both day 93 and day 105 after birth (with day 93 being more than 10 days after the treatment period had ended).

### EXAMPLE 2

### Parkinson's Disease

Parkinson's disease (PD) is characterized by the presence of aggregates (Lewy bodies) composed of alpha-synculein and other cellular proteins within neurons. (Paleologou; Gandhi) These aggregates are believed to be a critical factor in the death of the neuron and in the etiology of PD (Paleologou; Schrag). Mutated alpha-synuclein, as observed in those individuals with familial PD caused by synculein mutations, causes increased aggregate formation (Paleologou). A cell culture model is used to study alpha-synuclein aggregate formation in vitro. This model was created by using molecular biological techniques to transfect a dopaminergic cell line with DNA constructs that, when expressed, generate large amounts of normal alpha-synuclein (over-expressed) or mutated human alpha synuclein within the cells essentially as described by Perez et al (Perez). A control cell line was also generated that was transfected with empty vector, ie, did not express mutated alpha synuclein. These cells were then treated with Difluoromethylornithine (DFMO) to test the ability of this agent to reduce cell death.

As shown in Table 1, when compared to cells with empty vector only (Control cells), cells over-expressing alpha synuclein or expressing mutated alpha synuclein showed increased cell death. Compared to control cells, treatment with DFMO (100-500 uM) increased survival in cells over-expressing alpha synuclein and in cells expressing mutated alpha synuclein by up to 54%. Since the cells that die in humans with PD are dopaminergic neurons (that is, use dopamine as their neurotransmitter), these data from a dopaminergic neuronal cell line provide evidence that DFMO is efficacious in reducing neuronal death.

TABLE 1

| | | | % Dead Cells | % rescue |
|---|---|---|---|---|
| | | | minus | compared to |
| | % Dead | % Dead | % DFMO- | vector control |
| | Cells | Cells | treated cells^{#} | for DFMO |
| Construct | (untreated) | (DFMO) | (100 um) | treated cells |
| Vector only | 39.8% | 30.2 | 9.6% | |
| Alpha- | 44.8 | 29.8 | 15% | 54% |
| synuclein* | | | | |
| Mutated alpha | 51.6 | 38.1 | 13.5 | 40% |
| synuclein** | | | | |
| #Represents % cells rescued by DFMO. | | | | |
| *Human alpha synuclein (wild type) cDNA was amplified by polymerase chains reaction (PCR) and cloned into pcDNA 3.1 vector (purchased from Invitrogen). HA tag was fused to the alpha synculein C-terminus by adding the HA sequence in the PCR reverse primer. Cells were transfected with Fugene 6 Transfection Reagent (purchased from Roche Applied Science) and co-transfected with | | | | |
| # synphilin-1 plasmid (gift of Dr. Pamela McLean, Mass General Hospital East). Estimated transfection efficiency = 20% | | | | |

**Human A53T alpha-synuclein mutant and wild type alpha-synuclein were amplified by PCR and cloned into pEGFP-N2 vector (purchased from Clontech) to make a fusion protein. Cells were transfected as above.
MN9D cells were provided by Ruth Perez (University of Pittsburg) and were originally made by Alfred Heller and Lisa Won, University of Chicago.
Cell death was determined using Propidium iodide and cells counted using flow cytometry.

References:

Schrag A, Schott J M. Lancet Neurol. 2006 April; 5(4):355-63 Epidemiological, clinical, and genetic characteristics of early-onset parkinsonism.

Paleologou K E, Irvine G B, El-Agnaf O M. Biochem Soc Trans. 2005 November; 33(Pt 5):1106-10 Alpha-synuclein aggregation in neurodegenerative diseases and its inhibition as a potential therapeutic strategy.

Gandhi S, Wood N W. Hum Mol Genet. 2005 Oct. 15; 14 Molecular pathogenesis of Parkinson's disease.

Perez R G, Waymire J C, Lin E, Liu J J, Guo F, Zigmond M J. J Neurosci. 2002 Apr. 15; 22(8):3090-9. A role for alpha-synuclein in the regulation of dopamine biosynthesis.

The foregoing is illustrative of the present invention, and is not to be construed as limiting thereof. The invention is defined by the following claims, with equivalents of the claims to be included therein.

What is claimed is:

1. A method of treating a neurodegenerative disease in a subject in need thereof, comprising:
(a) orally administering said subject an ornithine decarboxylase inhibitor or a pharmaceutically acceptable salt or prodrug thereof in an amount effective to treat said disease; and concurrently
(b) orally administering said subject a polyamine or a pharmaceutically acceptable salt or prodrug thereof in an amount effective to reduce damage to the gastroinestinal tract of said subject by said ornithine decarboxylase inhibitor or pharmaceutically acceptable salt or prodrug thereof.

2. The method of claim 1, wherein said subject is a human subject.

3. The method of claim 1, wherein said neurological disease is a polyglutamine repeat disease.

4. The method of claim 4, wherein said polyglutamine repeat disease is selected from the group consisting of Huntington's disease, dentatorubral pallidoluysian atrophy, spinobulbar muscular atrophy, and spinocerebellar ataxia types 1, 2, 3, 6 and 7.

5. The method of claim 1, wherein said neurodegenerative disease is a amyotrophic lateral sclerosis.

6. The method of claim 1, wherein said ornithine decarboxylase inhibitor is alpha-difluoromethylornithine or a pharmaceutically acceptable salt or prodrug thereof.

7. The method of claim 1, wherein said polyamine is selected from the group consisting of spermine, spermidine, putrescine (1,4-diamino-butane), 1,3-diamino-propane, 1,7-diamino-heptane, and 1,8-diamino-octane.

8. The method of claim 1, wherein said administering steps are carried out chronically for at least six months.

9. The method of claim 1, wherein said administering steps are carried out chronically for at least one year.

10. A method of treating a neurodegenerative disease in a subject in need thereof, comprising simultaneously orally administering in a single oral dose formulation:
(a) an ornithine decarboxylase inhibitor or a pharmaceutically acceptable salt or prodrug thereof in an amount effective to treat said disease; and
(b) a polyamine or a pharmaceutically acceptable salt or prodrug thereof in an amount effective to reduce damage to the gastroinestinal tract of said subject by said ornithine decarboxylase inhibitor.

11. The method of claim 10, wherein said subject is a human subject.

12. The method of claim 10, wherein said neurodegenerative disease is a polyglutamine repeat disease.

13. The method of claim 11, wherein said polyglutamine repeat disease is selected from the group consisting of Huntington's disease, dentatorubral pallidoluysian atrophy, spinobulbar muscular atrophy, and spinocerebellar ataxia types 1, 2, 3, 6 and 7.

14. The method of claim 10, wherein said neurodegenerative disease is a amyotrophic lateral sclerosis.

15. The method of claim 10, wherein said ornithine decarboxylase inhibitor is alpha-difluoromethylornithine or a pharmaceutically acceptable salt or prodrug thereof.

16. The method of claim 10, wherein said polyamine is selected from the group consisting of spermine, spermidine, putrescine (1,4-diamino-butane), 1,3-diamino-propane, 1,7-diamino-heptane, and 1,8-diamino-octane.

17. The method of claim 10, wherein said administering step is a chronic administering step.

18. The method of claim 10, wherein said administering step is carried out chronically for at least six months.

19. The method of claim 10, wherein said administering step is carried out chronically for at least one year.

20. An oral dose formulation for treating a neurodegenerative disease comprising, in combination:
(a) an ornithine decarboxylase inhibitor or a pharmaceutically acceptable salt or prodrug thereof in an amount effective to treat said disease; and
(b) a polyamine or a pharmaceutically acceptable salt or prodrug thereof in an amount effective to reduce damage to the gastroinestinal tract of said subject by said ornithine decarboxylase inhibitor or pharmaceutically acceptable salt or prodrug thereof.

21. The formulation of claim 20, wherein said ornithine decarboxylase inhibitor is alpha-difluoromethylornithine or a pharmaceutically acceptable salt or prodrug thereof.

22. The formulation of claim 20, wherein said polyamine is selected from the group consisting of spermine, spermidine, putrescine (1,4-diamino-butane), 1,3-diamino-propane, 1,7-diamino-heptane, and 1,8-diamino-octane.

23. The formulation of claim 20, wherein:
said ornithine decarboxylase inhibitor or pharmaceutically acceptable salt thereof is included in said formulation in an amount effective to deliver a dosage thereof of 0.1 to 5 grams per meter² per day to said subject; and
said polyamine is included in said formulation in an amount effective to deliver a dosage thereof of 20 to 2000 mg per kg per day to said subject.

24. The formulation of claim 20 in the form of a tablet.

25. The formulation of claim 20 in the form of a capsule.

26. The formulation of claim 20 in the form of a liquid.

27. A method of treating Parkinson's disease in a subject in need thereof, comprising:
(a) orally administering said subject an ornithine decarboxylase inhibitor or a pharmaceutically acceptable salt or prodrug thereof in an amount effective to treat said Parkinson's disease; and concurrently
(b) orally administering said subject a polyamine or a pharmaceutically acceptable salt or prodrug thereof in an amount effective to reduce damage to the gastroinestinal tract of said subject by said ornithine decarboxylase inhibitor or pharmaceutically acceptable salt or prodrug thereof.

28. The method of claim 27, wherein said subject is a human subject.

29. The method of claim 27, wherein said ornithine decarboxylase inhibitor is alpha-difluoromethylornithine or a pharmaceutically acceptable salt or prodrug thereof.

30. The method of claim 27, wherein said polyamine is selected from the group consisting of spermine, spermidine, putrescine (1,4-diamino-butane), 1,3-diamino-propane, 1,7-diamino-heptane, and 1,8-diamino-octane.

31. The method of claim 27, wherein said administering steps are carried out chronically for at least six months.

32. The method of claim 27, wherein said administering steps are carried out chronically for at least one year.

33. A method of treating Parkinson's disease in a subject in need thereof, comprising simultaneously orally administering in a single oral dose formulation:
(a) an ornithine decarboxylase inhibitor or a pharmaceutically acceptable salt or prodrug thereof in an amount effective to treat said Parkinson's disease; and
(b) a polyamine or a pharmaceutically acceptable salt or prodrug thereof in an amount effective to reduce damage to the gastroinestinal tract of said subject by said ornithine decarboxylase inhibitor.

34. The method of claim 33, wherein said subject is a human subject.

35. The method of claim 33, wherein said ornithine decarboxylase inhibitor is alpha-difluoromethylornithine or a pharmaceutically acceptable salt or prodrug thereof.

36. The method of claim 33, wherein said polyamine is selected from the group consisting of spermine, spermidine, putrescine (1,4-diamino-butane), 1,3-diamino-propane, 1,7-diamino-heptane, and 1,8-diamino-octane.

37. The method of claim 33, wherein said administering step is a chronic administering step.

38. The method of claim 33, wherein said administering step is carried out chronically for at least six months.

39. The method of claim 33, wherein said administering step is carried out chronically for at least one year.
